(19) United States
(12) Patent Application Publication
(10) Pub. No.: US 2006/0216765 A1
(43) Pub. Date: Sep. 28, 2006

(54) Apparatus, methods and systems for rapid microbial testing

(76) Inventors: Thomas H. Grove, Manhattan Beach, CA (US); Stephan G. Thompson, El Segundo, CA (US); Donald S. Wong, Granada Hills, CA (US)

Correspondence Address: TRASK BRITT, P.O. BOX 2550, SALT LAKE CITY, UT, 84110, US

(21) Appl. No.: 11/091,017

(22) Filed: Mar. 25, 2005

(51) Int. Cl. G01N 33/554; C12Q 1/04

(52) U.S. Cl. 435/7.32; 435/34

(57) ABSTRACT

A method for detecting microorganisms includes obtaining a sample from a source of interest and applying the sample to a selective growth medium to permit any microorganisms of interest in the sample to grow. Any microorganisms that have grown are harvested. An immunoassay is then conducted to detect the microorganism of interest, if any, with a high degree of sensitivity and specificity. A system that may be used to grow, harvest, and detect a microorganism of interest includes a growth plate, or dish, a lavage solution, a lavage apparatus, and an assay apparatus. The dish may include a vessel with multiple sections, at least one of which is used to grow microorganisms of interest, and another of which is configured to receive and contain harvested microorganisms of interest.

### BACKGROUND OF THE INVENTION

1. Field of the Invention

The present invention relates generally to microbial testing techniques and, more specifically, to techniques for quickly and directly detecting microbes. Particularly, the present invention relates to microbial testing techniques in which a sample is obtained, microbes, if any, in the sample are grown, and, before or after microbial growth is visible with the naked eye, any microbes that have been grown are subject to a biological assay for a direct, sensitive, specific, and accurate indication of the presence or absence of one or more microorganisms of interest in the sample and, optionally, an indicator of the amount of such microorganisms that were present in the sample.

2. Background of Related Art

When surfaces become contaminated with bacteria, fungi, molds, yeasts, viruses, or other microorganisms, or “microbes,” sickness (morbidity) and, sometimes, death (mortality) may result. This is particularly true when surfaces in food processing plants and healthcare facilitates (e.g., hospitals) become contaminated with microorganisms.

In food processing plants, surfaces (e.g., solid surfaces, equipment surfaces, protective clothing, etc.) may become contaminated. Such contamination may be caused by or transferred to meat or other foods. In healthcare facilities, microbes may be released onto surfaces (e.g., solid surfaces, equipment surfaces, clothing, etc.) from infected individuals or otherwise. Once a surface becomes contaminated with microbes, contact with the contaminated surface may easily and readily transfer microbes to other locations, such as another surface, an individual, equipment, food, or the like.

As is well known, microbial contamination and transfer in certain environments may pose significant health risks. For example, the food that leaves a contaminated food processing plant will subsequently be eaten, and may cause sickness and, possibly, death. Microorganisms such as Listeria monocytogenes and Escherichia coli 0157:H7 are of particular concern. L. monocytogenes grows even when refrigerated, while E. coli O157:H7 infections are aggressive and often deadly.

Microbial contamination is of concern in healthcare facilities since some of the patients of such facilities often suffer from infections by pathogenic microbes and, thus, bring the pathogenic microbes into such facilities. Further, many of those who are present in such facilities (e.g., patients) are sick and may be immunologically compromised. These individuals are, thus, at increased risk of becoming sick from infection by the contaminating microbes.

In view of the potential dangers of microbial contamination, in particular the ease with which microbes may be transferred in certain environments and the health hazards associated with the contamination of certain environments, a variety of techniques have been developed and employed to detect such contamination so that it may be promptly remedied.

Conventionally, environmental microbial testing includes obtaining a sample from a surface. This is typically done by contacting (e.g., wiping, swiping, etc.) the surface with a sterile sampling appliance, such as a swab or a sponge. Surfaces that are tested in this manner are usually quite clean; thus, the number of microorganisms that are picked up by the sampling appliance is typically quite low. Due to the small sample size, any microbes that are on (e.g., picked up by) the sampling appliance must be reproduced, or “grown” or “cultured,” to provide a sufficient number of organisms that are suitable for further analysis. Accordingly, the sample is then typically neutralized and, optionally, stabilized, repaired, or enriched, then applied (e.g., swiping, dipping and agitating, etc.) to an appropriate growth media (e.g., agar (a gelatin or gelatin-like material), broth (a liquid), etc.), which includes nutrients that will help microbes of interest grow. The growth media may be selective, meaning that the growth media may include ingredients that will allow some microorganisms to grow at much faster rates than other microbes or ingredients that will prevent the growth of at least some undesired microbes. The growth media is incubated or held at a certain temperature for a predetermined period of time—typically about 24 to about 48 hours—or until microbial growth is visibly apparent.

Once the sample has had a sufficient opportunity to grow, the amount of bacteria (e.g., the number of colonies on an agar plate) that has grown may then be evaluated (e.g., by an individual or with automated equipment) to provide some indication of the number and type of microbes that were present on a certain area of the surface at the time the sample was taken-usually a day or two earlier. Immunological or other testing may also be performed to determine or confirm the identity or identities of any microbes of interest that were present in the sample.

For example, when testing for a Listeria species of bacteria, a sample potentially including the Listeria species may be applied to a selective growth media. The selective growth media may then be incubated for a period of about 24 to about 48 hours until growth of Listeria microbes is visible. Once Listeria colonies are visibly present on the selective growth media, the colonies may be evaluated to confirm their identities, and, optionally, counted to estimate a number of Listeria microorganisms present on a certain area of the tested surface. Alternatively or additionally, the cultured microorganisms may be subjected to an immunoassay or nucleic acid assay to more directly confirm their identities.

Conventional assay techniques that have the desired levels of specificity and sensitivity require additional, valuable time. For example, it takes an additional hour or two to conduct an enzyme-linked immunosorbent assay (“ELISA”) and up to four hours to conduct a polymerase chain reaction (“PCR”)-based assay for nucleic acid identification.

The long periods of time required by conventional microbial growth and testing techniques are somewhat undesirable since they typically do not provide sufficient time for an effective response to the potential transfer of and infection by contaminating microbes.

There is, therefore, a need for more rapid microbial assay techniques that have a desired level of sensitivity (i.e., can detect very small amounts of a microorganism of interest) and specificity (i.e., microorganisms other than those of interest do not generate a positive result).

### SUMMARY OF THE INVENTION

The present invention includes growth and assay techniques that may be used to determine whether or not one or more microorganism of interest is present in less time than that required by conventional processes.

An exemplary embodiment of a growth process according to the present invention includes obtaining a sample that may include at least one microorganism of interest, increasing a population of, or growing or proliferating, at least the microorganism of interest, if any, and harvesting a solution potentially including the at least one microorganism of interest before growth thereof is visibly evident (e.g., to the naked eye). Thereafter, the harvested solution may be assayed to determine whether or not the at least one microorganism is present therein and, thus, was present in the sample.

The present invention also includes a system and apparatus that are useful therein. An exemplary system includes microbial growth apparatus (e.g., growth plates, dishes, tubes, flasks, etc.), a lavage solution, a lavage appliance, and an assay apparatus.

An exemplary embodiment of growth apparatus comprises a dish, which is also referred to in the art as a plate, and a lid, or cover, for the dish. The growth apparatus includes a vessel that is separated into multiple sections, one of which is configured to contain a growth medium or media (e.g., a semisolid growth medium, such as a nutrient agar). A sample that potentially includes one or microorganisms of interest may be applied to a surface of the growth medium or media, which facilitates reproduction of the one or more microorganisms to better facilitate evaluation of the sample and, thus, of the potential contaminants of the source from which the sample was obtained.

At least one other section of the vessel is configured to receive lavage solution, which is also referred to herein as a “wash solution,” and microorganisms present therein following incubation and washing of the growth medium or media.

The lavage solution is configured for use in both harvesting microorganisms and facilitating a specific-binding assay.

The lavage appliance, when used in conjunction with the lavage solution, facilitates removal of microorganisms from the growth medium and their introduction into the lavage solution, as well as transfer of the lavage solution and microorganisms to a separate section of the vessel of the growth apparatus. The lavage solution may then be evaluated, using a suitable assay apparatus, to determine whether the one or more microorganisms of interest were present in the sample and, thus, on or in the source from which the sample was obtained. Evaluation of the lavage solution may also provide information about the number of microorganisms of interest that were present in (e.g., per unit volume) or on (e.g., per unit surface area) the sample source at the time the sample was obtained.

Other features and advantages of the invention will become apparent to those of ordinary skill in the art through consideration of the ensuing description, the accompanying drawings, and the appended claims.

### BRIEF DESCRIPTION OF THE DRAWINGS

In the drawings, which illustrate various aspects of exemplary embodiments of the inventive subject matter disclosed herein:

FIG. 1 schematically depicts a growth and diagnosis system that incorporates teachings of the present invention;

FIGS. 2A through 2C depict an exemplary embodiment of dish, with FIG. 2A showing the dish in perspective, with a cover in a partially open position thereover, FIG. 2B showing a top view of the dish with the cover in place thereover, and FIG. 2C comprising a cross-section taken along line 2C-2C of FIG. 2B;

FIG. 3 illustrates an exemplary lavage appliance;

FIG. 4 shows an example of introduction of a semisolid growth media into a vessel of a dish;

FIG. 5 shows a cover in place over a dish with semisolid growth media therein;

FIG. 6 schematically illustrates an exemplary manner of obtaining a sample;

FIG. 7 shows repair of the sample;

FIG. 8 depicts an example of application of a sample to a surface of a quantity of semisolid growth media;

FIG. 9 shows another example of application of a sample to a surface of a quantity of semisolid growth media;

FIG. 10 illustrates introduction of a reagent solution, such as a lavage solution, onto a surface of a quantity of semisolid growth media that potentially includes microorganisms of interest growing thereon;

FIG. 11 depicts an exemplary manner in which microorganisms present on the surface of the quantity of semisolid growth media may be dispersed throughout the reagent solution;

FIGS. 12 and 13 illustrate collection of the reagent solution for evaluation; and

FIG. 14 is a flow diagram illustrating an exemplary technique for determining whether or not a particular microorganism is present in a sample.

### DETAILED DESCRIPTION

The apparatus and system of the present invention may be used for a variety of applications, including, but not limited to, food testing, water testing, medical and veterinary testing and evaluation, and forensic testing. The apparatus and system of the invention, as well as the inventive methods, may be used to determine, qualitatively (present or not?) or quantitatively (an amount), the presence of one or more microorganisms of interest.

By way of nonlimiting example, teachings of the present invention may be used to determine whether bacteria, fungi, molds, yeasts, viruses, or other microorganisms are present in a sample. The systems, apparatus, and methods of the present invention may, for example, be used to determine whether bacteria of one or more of the genuses Listeria, Campylobacter, Escherichia, Salmonella, Clostridia, Shigella, Staphylococcus, Vibrio, Yersinia, Plesiomonas, Bacillus, Streptococcus, Neisseria, Enterococcus, Enterobacter, Citrobacter, Vibrio, Legionella, Haemophilus, Pseudomonas, Gardnerella, Francisella, Brucella, Bordetella, Borrelia, Mycobacterium, Nocardia, and Aeromonas is present in a sample. Samples may be assayed for the presence of one or more yeasts, including, without limitation, yeasts of one or more of the genuses Kluyveromyces, Pichia, Saccharomyces, Candida, and Rhodotorula. Alternatively, or in addition, samples may be evaluated for molds, including, but not limited to, molds of the genuses Byssochlamys, Fusarium, Geotrichium, Penicillum, Aspergillosis, and Scopulariopsis.

### Apparatus and Systems

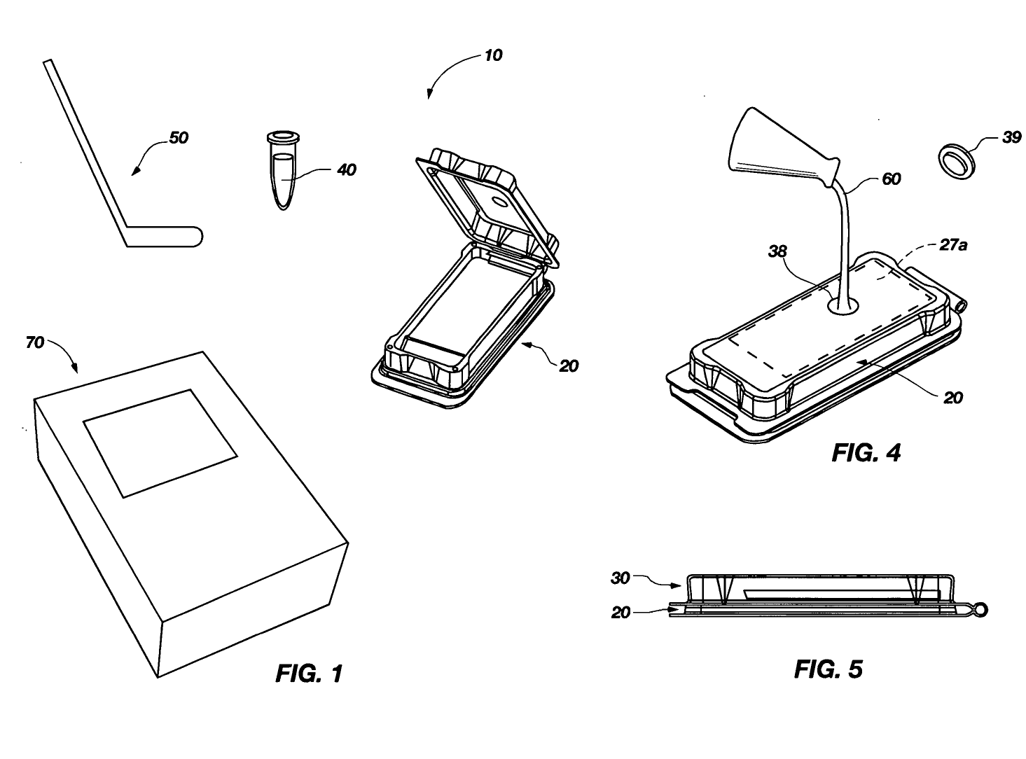

FIGS. 1 though 3 depict an exemplary assay system 10 that incorporates teachings of the present invention, as well as various apparatus and other components of such a system. An example of an assay technique that incorporates teachings of the present invention is illustrated in FIGS. 4 through 13.

The features of an exemplary growth and detection system 10 according to the present invention are shown in FIG. 1. System 10 includes a dish 20, a lavage solution 40, a lavage appliance 50, and an assay apparatus 70.

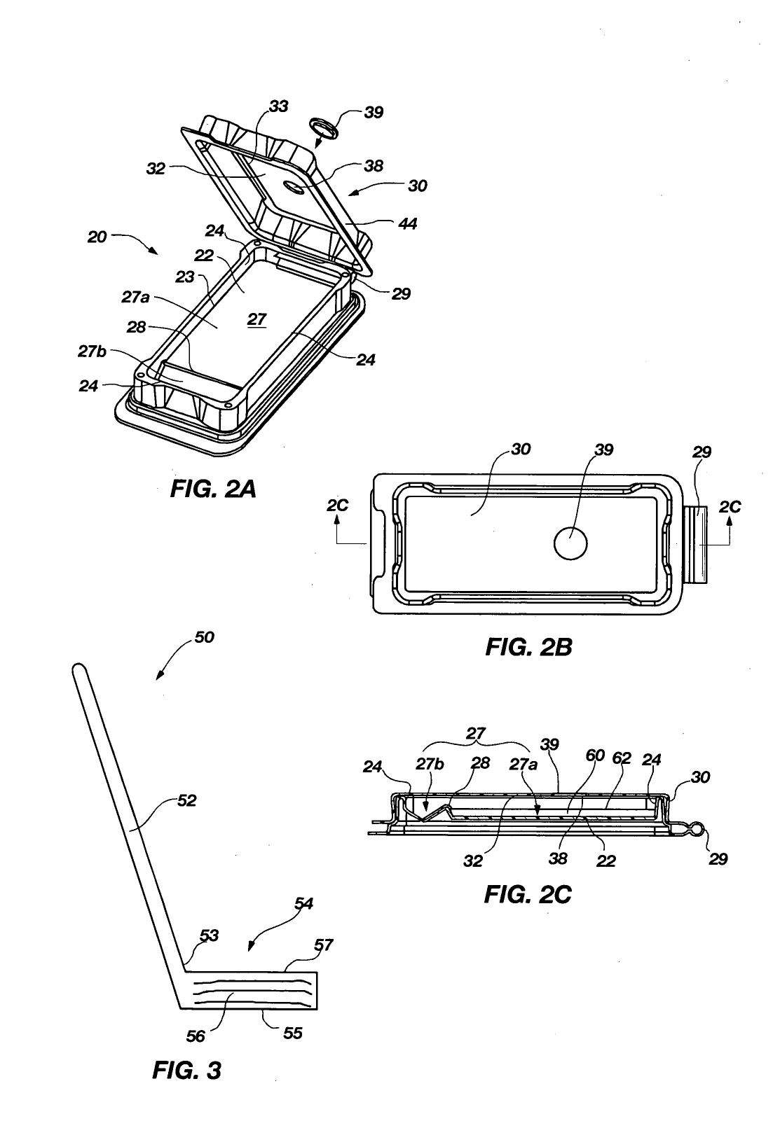

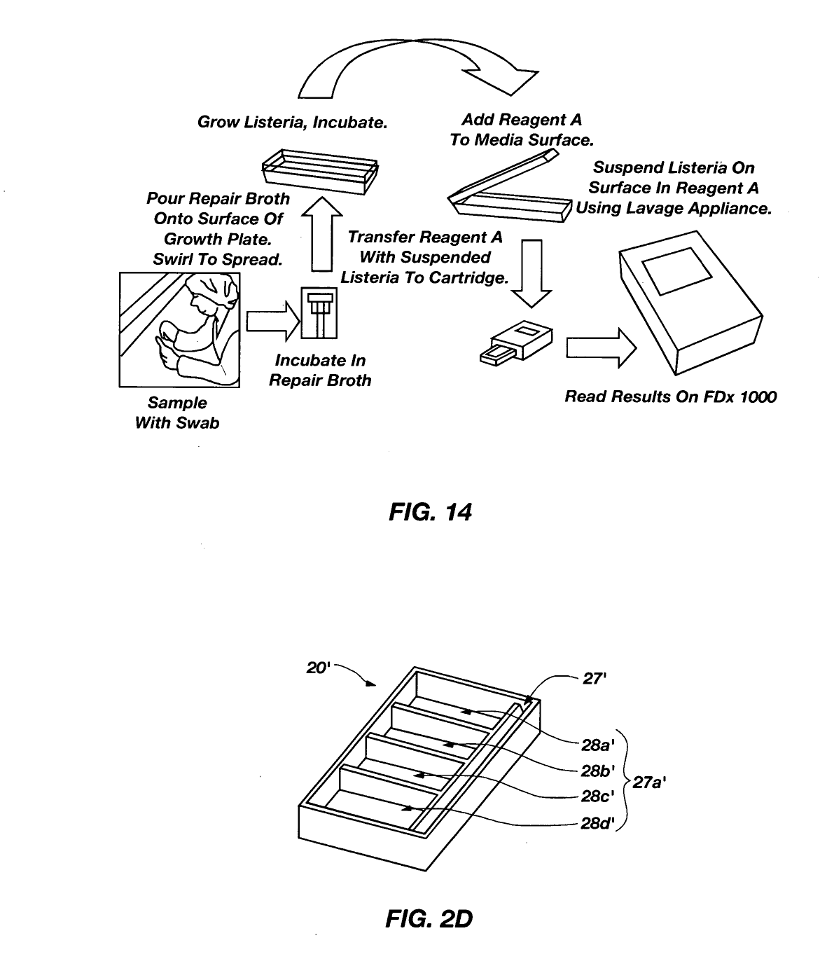

As shown in FIGS. 2A through 2C, an exemplary growth apparatus, a dish 20, that incorporates teachings of the present invention includes a vessel 27. Vessel 27 is defined by a base 22 and sidewalls 24 of dish 20. As shown, base 22 may be substantially planar. Sidewalls 24 extend upwardly from the peripheral edges 23 of base 22. Vessel 27 is separated into a plurality of sections 27a, 27b. One or more separation walls 28 extend across vessel 27 to laterally isolate different sections 27a, 27b thereof from one another.

The larger of the vessel sections 27a shown in FIGS. 2A and 2C may, by way of nonlimiting example, be used to contain a growth medium. By way of example only, the growth medium within vessel section 27a may be a semisolid growth medium 60, such as a nutrient, or enrichment, agar or another gelatinous material. Semisolid growth medium 60 may be configured, as known in the art, to selectively promote the growth of only one or a few microorganisms of interest. For example, MacConkey Sorbitol agar, C-T supplement, Rainbow agar, BCM agar, CHROMagar 0157, and 202 Agar are known to be useful for facilitating the proliferation of Escherichia coli O157:H7 with some selectivity. Oxford agar, modified Oxford agar, PALCAM agar, and Aloa agar are examples of semisolid growth media that facilitate the selective growth of L. monocytogenes. Staphylococcus aureus may be grown with some selectivity on 110 agar, Baird-Parker agar, and Mannitol Salt agar, to name only a few. Campylobacter agars and blood-free Campylobacter agars are examples of semisolid growth media that may be used to facilitate the selective growth of bacteria of the Campylobacter genus. Exemplary agars that facilitate the selective growth of bacteria of the genus Clostridium include, without limitation, Sulfite Polymyxin Sulfadiazine (“SPS”) agar and Shahidi-Ferguson-perfringens (“SFP”) agar. Bacteria from the genus Vibrio may be selectively grown on Thiosulfate-Citrate-Bile-Sucrose (“TCBS”) Cholera agar, a charcoal gelatin disc, or Cholera agar. A variety of agars, including, without limitation, Xylose Lysine Desoxycholate agar, Xylose Lysine Tergitol 4 agar, Brilliant Green agar, Mannitol Lysine Crystal Violet Brilliant Green (“MLCB”) agar, modified semisolid Rappaport-Vassiliadis (“MSRV”) agar, Kligler Iron agar, Rappaport Vassiliadis Soya agar, Lysine Iron agar, Lysine Desoxycholate agar, Rambach agar, Simmons Citrate agar, CHROMagar Salmonella, Triple Sugar Iron agar, Hektoen Enteric agar, Bismuth Sulphite agar, and Salmonella Shigella (“SS”) agar, may be used to grow bacteria from the genus Salmonella with some selectivity. Bacteria from the genus Enterobacteriaceae may be grown with some selectivity on a variety of semisolid growth media, including, but not limited to Violet Red Bile (“VRB”) agar, Eosin Methylene Blue (“EMB”) agar, Sulfur-Indole-Motility (“SIM”) agar, Brilliant Green Bile agar, Lauryl Tryptose agar, Simmons Citrate agar, Triple Sugar Iron agar, Desoxycholate agar, Desoxycholate Lactose agar, Levine agar, and Kligler Iron agar. Of course, agars or other semisolid growth media that selectively or nonselectively support the growth of a variety of other types of microorganisms may also be used with a dish 20 of the present invention and in accordance with other teachings of the present invention.

The smaller of the vessel sections 27b shown in FIGS. 2A and 2C may be used to receive a wash solution, such as a quantity of lavage solution 40 (FIGS. 1 and 13) that has been applied to surface 62 of semisolid growth media 60 to remove microorganisms therefrom, including any microorganisms of interest therein.

Another exemplary embodiment of a dish 20′ of the present invention, shown in FIG. 2D, is configured to grow a number of different types of microorganisms simultaneously. In particular, vessel 27′ of dish 20′ includes a plurality of growth areas 28a′, 28b′, 28c′, etc. that are configured to contain different types of growth media, each of which facilitates the selective growth of a particular type of microorganism. Growth areas 28a′, 28b′, 28c′, etc. may comprise different areas of a single growth section 27a′ of vessel 27′, or separate sections of vessel 27′.

A lid 30 is also shown in FIGS. 2A through 2C. Lid 30 is configured to be assembled with dish 20 in such a way as to facilitate sterilization of vessel 27 and its contents, as well as to prevent subsequent contamination of vessel 27 and its contents. Although illustrated as being secured to dish 20 with a hinge 29, lid 30 may be separate from dish 20.

Lid 30 includes a ceiling 32, which may be substantially planar, as well as peripheral walls 44 extending downwardly from the peripheral edges 33 of ceiling 32. When positioned over culture dish 20, lid 30 and peripheral walls 24 of culture dish 20 provide a somewhat convoluted path, which may facilitate sterilization of vessel 27 and its contents, as well as the introduction of gases into and the exhaustion of gases from vessel 27, while undesired microorganisms are prevented by lid 30 and peripheral walls 24 of culture dish 20 from entering and, thus, contaminating vessel 27.

One or more small access holes 38 (only one is shown) may be formed in ceiling 32 of lid 30 to provide access to one or more parts of vessel 27 without requiring that a large portion of vessel 27 and its contents be exposed to the external environment and, thus, potential contaminants. Access hole 38 may be covered with and retain a complementary access lid 39.

Dish 20 and lid 30 are formed from substantially nonporous, substantially inert materials that will withstand sterilization processes. As a nonlimiting example, dish 20 and lid 30 may be manufactured from a plastic (e.g., polystyrene, PETG), glass, or the like. Of course, inexpensive materials are desirable for dishes 20 and lids 30 that are intended for a single use (i.e., that are disposable), while more durable materials are desirable for reusable dishes 20 and lids 30.

With returned reference to FIG. 1, lavage solution 40 may include sterile deionized water, buffer, salts, detergent, rate accelerators (e.g., polyethylene glycol), lysing agents, and a component that binds specifically to one or more microorganisms of interest. The specific-binding component may, by way of example only, comprise an antibody or antibodies. As another example, the specific binding component may comprise an oligonucleotide. The specific binding component of lavage solution 40 may be labeled, directly or indirectly (e.g., by way of another antibody or oligonucleotide), with a detectable marker, such as a fluorogenic moiety, phosphorescent moiety, radioactive moiety, chemiluminescent moiety, nephelometric moiety, electrochemiluminescent moiety, bioluminescent moiety, enzyme moiety, latex bead moiety, or the like.

Lavage appliance 50 is shown in FIG. 3. Lavage appliance 50 includes an elongate handle 52 and a massage element 54.

Handle 52 is configured to be grasped by the hand of an individual in such a way as to enable the individual to manipulate lavage appliance 50 and move massage element 54 as desired.

Massage element 54 extends from an end 53 of handle 52. As shown, massage element 54 is an elongate element that is oriented at an angle to handle 52. A bottom edge 55 of massage element 54 is configured to contact a surface 62 of semisolid media 60 (e.g., a growth agar, other gelatinous media, etc.) (FIG. 2C) without significantly disrupting the same. Bottom edge 55 is also configured to seal against the surface of the semisolid media 60 in such a way that liquid and organisms on the surface may be moved by massage element 54. At least one face 56 of massage element 54 is configured to push liquid and organisms across the surface of the semisolid media 60 without permitting a significant thereof from flowing over the top edge 57 of massage element 54.

Like dish 20 and lid 30 (FIGS. 2A through 2C), lavage appliance 50 may be formed from a substantially inert material that will withstand sterilization processes. By way of example, a single-use, or disposable, lavage appliance 50 may be formed from plastic, packaged, and sterilized in a suitable fashion so that, when use thereof is desired, it may be removed from its packaging or other containment in a sterile or substantially sterile state. A reusable lavage appliance 50 may be formed from a more durable material, such as metal, ceramic, or glass, that will withstand repeated sterilization and, optionally, that may be sterilized on the laboratory bench when use thereof is desired.

Assay apparatus 70 (FIG. 1) may comprise any assay system (e.g., an immunoassay system, a nucleic acid assay system, etc.) that operates with sufficient sensitivity and specificity to accurately determine whether or not a small amount of a microorganism of interest (e.g., a bacterium, fungus, mold, yeast, virus, or other microorganism) is present in a relatively dilute sample. For example, an instrumented assay system, such as the BioCentrex FDx 1000 Analyzer, a fluorescent waveguide assay apparatus manufactured by BioCentrex, LLC, of Culver City, Calif., may be used as assay apparatus 70. Alternatively, a noninstrumented assay device, such as a lateral flow device, an enzyme-linked immunosorbent assay (ELISA) system, or the like, may be employed, as may any other available immunoassay system, nucleic acid assay system, or other biological assay system.

### Techniques

Various aspects of a method that incorporates teachings of the present invention will now be described with reference to FIGS. 4 through 13. Although many of these techniques reference partial removal of lid 30 to access a portion of vessel 27 or a surface 62 of semisolid growth media therein, in some instances, such access may alternatively be achieved by removing an access lid 39 from an access opening 38 (see FIGS. 2A through 2C, 4).

In FIG. 4, a quantity of semisolid growth media 60 is prepared, as known in the art and, while in a liquid state, introduced into section 27a of vessel 27 (FIGS. 2A and 2C) of dish 20 (e.g., through access hole 38, as shown, or by lifting lid 30). A lid 30 is positioned over dish 20, as shown in FIG. 5. Thereafter, dish 20 and its contents (e.g., semisolid growth media 60 are sterilized by a technique suitable for use with semisolid growth media 60 and the material or materials from which dish 20 and lid 30 are formed. Dish 20 may then be stored in a suitable manner and for an acceptable duration, as known in the art to be appropriate for the type of semisolid growth media 60 within section 27a of vessel 27.

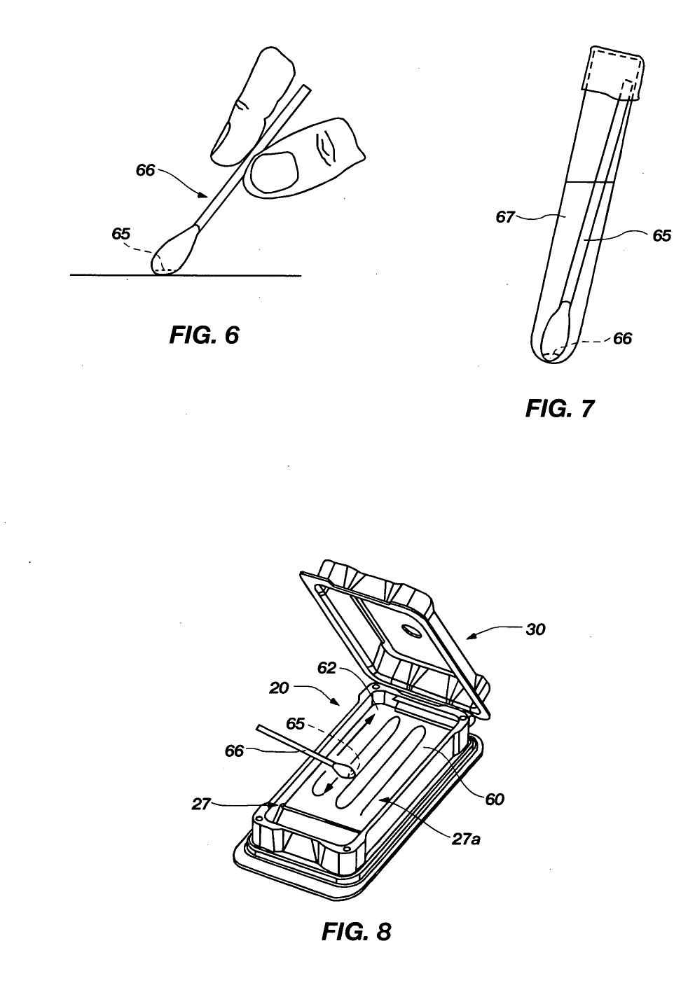

As illustrated in FIG. 6, a sample 65 may be collected, as known in the art and in a manner that is suitable for obtaining a sample from a particular source. Sample 65 may, by way of nonlimiting example, comprise a sample obtained from a surface, a food sample, a food surface sample (e.g., from a carcass), a liquid sample, (e.g., from water or dairy products), or an air sample. A sampling appliance 66 of a type known in the art, such as a swab or sponge, may be used to collect sample 65. The collection of sample 65 may be effected in such a way as to minimize contamination thereof.

Once sample 65 has been collected, undesirable components thereof (e.g., residual disinfectants) may be neutralized or any cells in the sample may be stabilized, pre-enriched, or repaired, as known in the art. For example, as shown in FIG. 7, sample 65 (and, optionally, sampling apparatus 66) may be held in a transitional liquid 67 (e.g., for 30 minutes to 48 hours). Examples of transitional liquid 67 include neutralizing buffers, such as Letheen buffer or D/E buffer, and nutrient broths, such as buffered peptone water or brain heart infusion broth. Transitional liquid 67 may be held at a temperature optimal for one or more microorganisms of interest that are potentially present in sample 65 to be neutralized, to stabilize themselves from the trauma associated with the sample-gathering process, to repair themselves, or to be enriched in preparation for subsequent growth.

When use of dish 20 and the semisolid growth media 60 therein is desired (e.g., when surface testing or other types of testing are desired), lid 30 is removed at least partially from dish 30 to provide access to section 27a and the semisolid growth media 60 therein, as shown in FIG. 8. Lid 30 is preferably manipulated in a manner that will prevent undesired contamination of surface 62 of semisolid growth media 60. For example, an edge of lid 30 may be lifted in such a way that lid 30 continues to cover or substantially cover vessel 27 of dish 20.

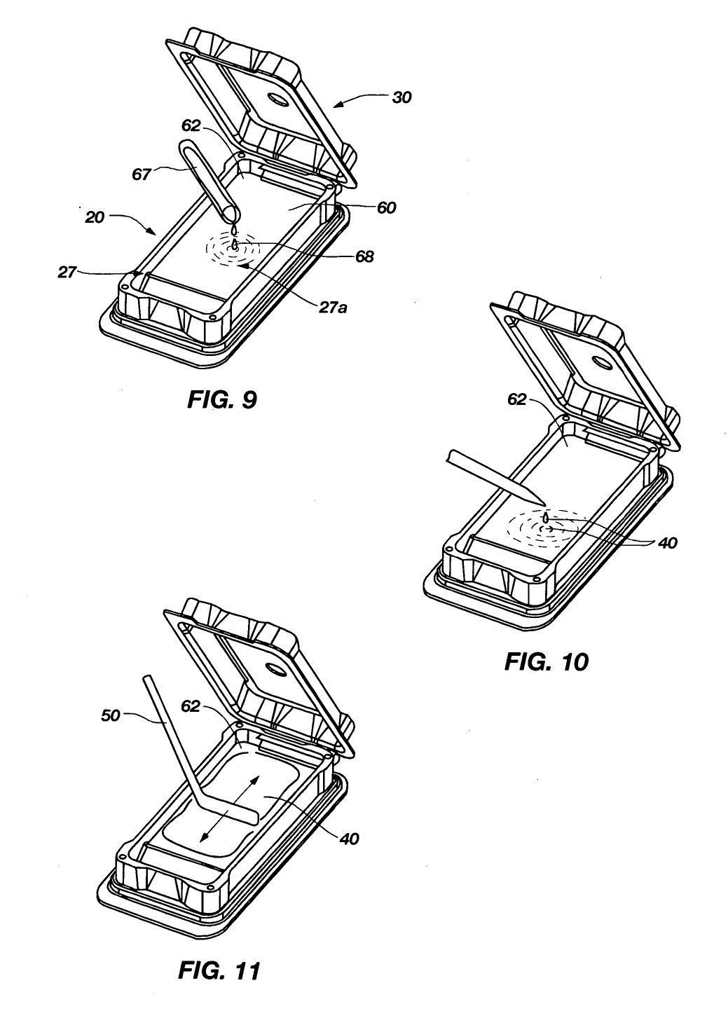

A sample 65 may then be applied to surface 62 by wiping surface 62 with a sampling appliance 66, such as a swab or sponge, by which sample 65 is carried. Alternatively, as illustrated in FIG. 9, a sample 68 of transitional liquid 67 may be transferred to surface 62 of semisolid growth media 60. By way of nonlimiting example, sample 68 may have a volume of about 50 μL to about 5 mL. Once sample 65, 68 has been applied to surface 62, lid 30 may be replaced over dish 20.

One or more microorganisms may be cultured within section 27a of vessel 27 of dish 20 (FIGS. 8 and 9) by incubating lid 30-covered dish 20 and its contents at an optimal or desired growth temperature for a specified period of time. Notably, the inventive methods include incubation for a period of time sufficient to permit proliferation of one or more microorganisms of interest without consuming unnecessary and valuable time that would otherwise be required to allow visible colonies or plaques of the one or more microorganisms of interest to be formed on surface 62 of semisolid growth media 60. Thus, the incubation time is shortened (e.g., to about one hour to about twenty-four hours).

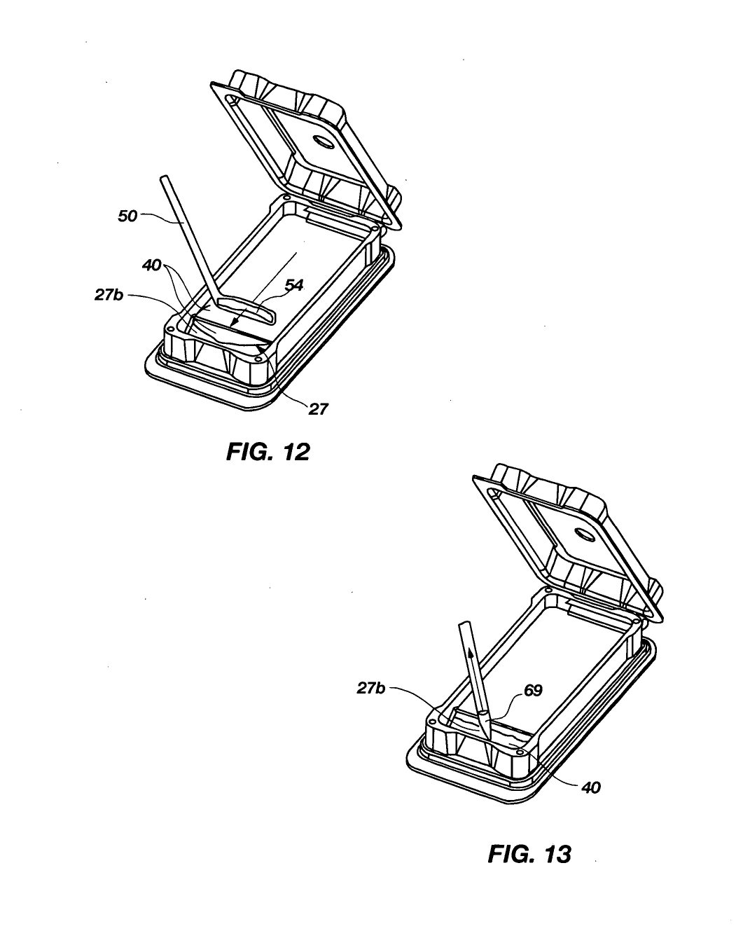

Once the incubation period has expired, lid 30 is again at least partially removed from dish 20 so as to provide access to surface 62 of semisolid growth media 60. As shown in FIG. 10, a quantity of lavage solution 40 (e.g., about 20 μL to about 1 mL) is then applied to surface 62, and surface 62 is massaged or otherwise manipulated in such a way (e.g., with lavage appliance 50, as shown; by ultrasonic vibration; etc.) as to facilitate the transfer of microorganisms from surface 62 to lavage solution 40, as shown in FIG. 11. Next, lavage solution 40 and any microorganisms therein are transferred to section 27b of vessel 27; for example, by pushing lavage solution 40 into section 27b with massage element 54 of lavage appliance 50, as illustrated in FIG. 12.

Thereafter, a sample 69 of lavage solution 40 is removed from section 27b and transferred, as known in the art and in a manner consistent with the use of a particular type of assay system 70, to assay system 70 (see FIG. 1), as is schematically depicted in FIG. 13. Sample 69 is then assayed, as known in the art, to determine whether or not the one or more microorganisms of interest are present and, optionally, to quantify the amount (e.g., concentration) of each of the one or more organisms that were present in the original sampling.

When a sample is being evaluated for the potential presence of multiple microorganisms, the individual assays for each microorganism may be conducted sequentially, simultaneously as separate assays, or simultaneously as multiplexed assays. For example, a test system may be configured to grow, harvest, and assay Salmonella and E. coli O157:H7 or any other combination of two or more microorganisms of interest simultaneously.

The examples that follow include the detection of Listeria, E. coli, and Salmonella and relate to bacteria obtained from environmental surfaces. The entire process for environmental Listeria testing is completed in less than 18 hours. FIG. 14 is a flow chart that summarizes an exemplary embodiment of a rapid surface testing process for detecting a bacteria of the genus Listeria (e.g., L. monocytogenes).

In addition, instead of immunoassay, nucleic acid testing can be performed on the lavaged sample from the agar plate.

### Listeria TESTING

### EXAMPLE 1

### Preparation of Immunoassays for Enviromental Listeria Testing

Affinity-purified goat antibody to Listeria obtained from Fitzgerald Industries International of Concord, Mass., was labeled with a 15-fold molar excess of Alexa-Fluor 647-succinimidyl ester from Molecular Probes, Inc., of Eugene, Oreg., in 100 mM sodium borate, pH 8.3, and 150 mM NaCl. The fluorescent conjugate was separated from the free dye by gel filtration through a column of 100 mL of Sephadex G-50 beads in a buffer including 50 mM Bis Tris Propane, pH 7.0, 150 mM NaCl, and 0.05% sodium azide. After spectral characterization, the conjugate was diluted to 7.2 mcg IgG/mL in a buffer including 150 mM Tris-HCl, pH 8.0, 54 mg bovine serum albumin (“BSA”)/mL, 18% sucrose, 300 mM NaCl, 0.1% Tween-20 and 5 mg of goat gamma globulin/mL. This conjugate solution was in turn diluted 1:2 with phosphate buffered saline, pH7.4, containing 0.05% Tween 20 (“PBST”). This final solution is referred to as “Reagent A.”

Affinity-purified goat anti-Listeria antibody was biotinylated by reacting the antibody with a 10-15 fold molar excess of EZ-Link™ NHS-LC-LAC-Biotin from Pierce Biotechnology, Inc., of Rockford, Ill., in 100 mM sodium borate, pH 8.3, and 150 mM NaCl. The biotinylated capture antibody was then separated from the unconjugated biotin derivative by desalting in Amicon® Centricon YM-30 centrifugation filter device, from Millipore Corporation of Billerica, Mass., into phosphate-buffered saline, pH 7.2.

Six μL of a 0.5 μM solution of the biotinylated antibody was applied to each spot of NeutrAvidin™ Biotin Binding Protein from Pierce Biotechnology, Inc., that had been previously applied to a surface of a BioCentrex polystyrene planar waveguide. The antibody-spotted waveguide was allowed to dry overnight at ambient temperature prior to use.

The immunoassays were performed by contacting a defined volume of Reagent A to an agar plate on which Listeria had been growing for a defined period of time at a defined temperature. Reagent A, which contained harvested Listeria, was collected in a portion of a plate accessible to a pipette tip or other transfer device, and a sample of about 100 μL to about 150 μL of the Reagent A-Listeria mixture was introduced into a waveguide-containing cartridge of the BioCentrex assay system. The sample-loaded cartridge was then placed in one of two analyzers.

The first analyzer that was employed was a BioCentrex portable analyzer equipped with a 635 nm laser, photodiodes, and a 660 nm longpass filter. Fluorescence that developed at the spot was monitored by the analyzer at 15 second intervals for a total duration of ten minutes. The binding rate recorded by the analyzer, as determined from changes in the measured fluorescence, was proportional to the concentration of Listeria harvested by Reagent A or a combination of the conjugate and another solution, such as PBST, which can be used to collect or dilute the cells.

The second analyzer was a BioCentrex benchtop analyzer equipped with a 658 nm red laser and a 703 nm bandpass filter. With the benchtop analyzer, the immunoassay binding reaction was monitored with a CCD camera for repeated two second exposure periods spaced at 6.5 second intervals. The entire exposure process lasted eight minutes.

### EXAMPLE 2

### Sample Collection and Preparation

Listeria sampling included placing a swab or small sponge previously wetted with the appropriate transitional liquid into contact with a defined area of a tested surface. The swab or sponge was then placed in a transitional liquid for a short period of time (e.g., about one hour to about four hours) at a defined temperature (e.g., room temperature). An aliquot of the liquid broth was then spread onto the surface of the appropriate semisolid growth media and microorganisms were allowed to proliferate at a defined temperature (e.g., 37° C.). After incubation for a defined duration (e.g., about 12 hours to about 18 hours), the cells were harvested and assayed.

### Example 3

### L. monocytogenes 4e Colonies Grown on Oxford and Brain Heart Infusion Agar

L. monocytogenes 4e present in the sample was grown on both Oxford and Brain Heart Infusion (BHI) agar plates for 17 hours at 37° C. Single colonies were sampled and dispersed into 0.3 mL of PBST and diluted 1/1 (“Neat”), 1/10, or 1/100, then mixed 2:1 with Reagent A and tested on the portable analyzer. In addition, a “Blank” was prepared by washing semisolid growth media to which samples had not been applied with the PBST, then adding the Reagent A to the PBST. The rates of binding between the L. monocytogenes 4e in each sample dilution and the antibodies on the waveguide are identified in TABLE 1.

TABLE 1

| Growth Media | Sample Dilution | Rate |
|---|---|---|
| BHI | Blank | −3 |
| | Neat | 2675 |
| | 10-fold | 903 |
| | 100-fold | 93 |
| Oxford | Blank | −1 |
| | Neat | 2560 |
| | 10-fold | 1994 |
| | 100-fold | 418 |

These data indicate that the immunoassay reaction is specific for Listeria, can be used to confirm the presence and identity of microorganisms selected from the surface of an agar plate, and are proportional to the bacterial concentration (by dilution), with similar results for both types of semisolid growth media.

### EXAMPLE 4

### L. monocytogenes 4e Introduced into BHI Broth or Buffered Peptone Water Before Growth on Oxford Agar

L. monocytogenes cells were delivered into 5 mL of respective BHI and buffered peptone water (BPW) repair broths to obtain a final concentration of either 18 or 180 cells/mL, then incubated for 2 hours or 4 hours at 37° C. A 1.0 mL aliquot of the repair broth was transferred to the surface of an Oxford plate and grown for 12 or 14 hours at 37° C. Cells on the entire surface of the agar were then harvested by scraping with a lavage appliance and dispersing the cells into 0.3 mL of Reagent A. The solution was then transferred to a Listeria immunoassay test cartridge and analyzed on the benchtop analyzer. Agar with no bacteria was also sampled and tested (“Blank”).

The results, in TABLE 2, show that two hours of repair in BPW and 14 hours on agar at 37° C. yielded higher binding rates for L. monocytogenes for both inoculums.

TABLE 2

| | |
|---|---|
| Inoculum | Repair |

| (Cells/mL) | Broth | Hours | Hours on Plate | Rate |
|---|---|---|---|---|
| 18 | BHI | 2 | 14 | 54 |
| | | 4 | 12 | 18 |
| | | 2 | 14 | 107 |
| | | 4 | 12 | 22 |
| 180 | BPW | 2 | 14 | 113 |
| | | 4 | 12 | 48 |
| | | 2 | 14 | 231 |
| | | 4 | 12 | 195 |
| Blank | | None | None | 2 |

### EXAMPLE 5

### Effect of Harvesting Different Numbers of Colonies by Immunolavage

Various dilutions of L. monocytogenes 4e were grown overnight on BHI agar plates at 37° C. Colonies were counted, then the cells were harvested and assayed with the benchtop analyzer. These results shown in TABLE 3 demonstrate the dose response.

TABLE 3

| Number of Colonies per Sampling | Rate |
|---|---|
| 5 | 41 |
| 10 | 107 |
| 100 | 406 |

In addition, various numbers of L. monocytogenes cells (i.e., 0, 50, and 100 cells) were added to separate, duplicate sterile vials containing 1 mL of buffered peptone water. The cells were allowed to repair for one hour at room temperature. After one hour, the entire 1 mL of each vial was poured onto the surfaces of separate Petri dishes containing Oxford agar. The plates were incubated at 37° C.

After 14 hours, one set of plates was removed from the incubator and inspected for colonies of L. monocytogenes cells. No L. monocytogenes colonies were seen at 14 hours. After inspection, the surfaces of the Oxford agar of these plates were washed with Reagent A and massaged. The Listeria immunoreactivity of the solution obtained from the surfaces of the Oxford agar of these plates was then determined.

After 24 hours, the duplicate set of plates was removed from the incubator and the number of Listeria colonies was counted on the surfaces of the Oxford agar plates. Listeria colonies appeared as black spots on the surfaces of the Oxford agar plates. No immunoassay was performed on these samples, as the appearance of colonies provided a reliable indicator of the number of microorganisms present in each of the initial samples.

The results of this experiment, which are set forth in TABLE 4, demonstrate a quantitative relationship between the number of starting cells, the number of colonies grown on the plates, and the L. monocytogenes immunoreactivity of the agar surface wash. While no Listeria colonies were seen at 14 hours, L. monocytogenes was clearly detected by the immunoassay.

TABLE 4

| | Colonies Observed | | Immunoassay Rate |
|---|---|---|---|
| Initial Innoculum | At 14 Hours | At 24 Hours | At 14 Hours |
| 0 | 0 | 0 | 0 |
| 50 | 0 | 12 | 18 |
| 100 | 0 | 35 | 28 |
| 100 | 0 | 49 | 35 |

### EXAMPLE 6

### Effect of Culture Time on Response of Wash Process

L monocytogenes cells, at a dilution of 280 cells/mL, were incubated in BPW repair broth for one hour at 37° C. Thereafter, 0.5 mL of the solution was transferred to each of four Oxford plates, which were then reincubated for various times (i.e., 12 hours, 14 hours, 16 hours, and 18 hours) at 37° C. The colonies were harvested from duplicate plates with Reagent A and tested on the portable analyzer. Results for this experiment are tabulated in TABLE 5 and show that the response is quantitative and dependent upon the culture incubation time.

TABLE 5

| Culture Time | Rate |
|---|---|
| 12 hours | 6.7 |
| 14 hours | 5.3 |
| 16 hours | 116 |
| 18 hours | 190 |

### EXAMPLE 7

### Simultaneous Reaction Using a Two-Component Reagent

An assay was performed using Reagent A, which included the AF647-labeled antibody, as well as 14.4 μg of biotinylated capture antibody per mL of conjugate solution, which is hereinafter referred to as “modified Reagent A.” This modified Reagent A was used to harvest cells from an agar plate and perform the immunoassay using an assay cartridge that contained a waveguide spotted only with NeutrAvidin in the manner described in EXAMPLE 1 above. A replicate test was also performed using unmodified Reagent A, in which the biotinylated capture antibody was not included. The results shown in TABLE 6 below demonstrate that both immunoassay components (capture and reporter antibody) can be included in a lavage solution, and that the response is dependent upon the presence of the biotinylated capture reagent.

TABLE 6

| Biotin-Antibody | Rate |
|---|---|
| Present (i.e., modified Reagent A) | 52 |
| Absent (Reagent A) | 6 |

### EXAMPLE 8

### Detection of Salmonella Nucleic Acids Using PCR

Salmonella choleraesuis was grown to stationary phase in nutrient broth overnight. The cells were 10-fold serially diluted from 10⁻⁵ to 10⁻⁸ in nutrient broth. A swab was then used to transfer cells from each dilution to 3 mL buffered peptone water. The cells were allowed to recover at 37° C. for one hour, then 0.5 mL of the culture was transferred onto a plate including nutrient agar. After incubation at 37° C. for 2, 4, and 8 hours, the cells were harvested by rinsing the plate surface with 300 μL of deionized water. Roughly 200 μL of agar wash, which included the deionized water and cells, was recovered.

The agar wash was then heated at 99° C. for 10 minutes. PCR was performed with 1 μL of the agar wash from each plate for 35 cycles. The amplicons were analyzed with a 2% agarose gel. The sample from 10⁻⁵ dilution that was cultured for eight hours yielded a high positive result. The rest of the samples were not detectable by the agarose gel.

The results of EXAMPLES 1 through 8 show that an environmental test for Listeria has been developed which is configured to determine if a surface was contaminated with Listeria in less than 18 hours. This time frame enables the test user to make informed decisions as to the efficacy of previous cleaning cycles and procedures, potential sources of contamination (i.e., by identifying the sample sources on which contamination is detected), and potential avenues for removing the contaminating microorganisms and preventing further contamination.

### Escherichia coli O157:H7 and Salmonella typhimurium Combined Test

### EXAMPLE 9

### Construction of the Immunoassays

AF647 fluorescence labeled and biotinylated conjugates for both E. coli O157:H7 and Salmonella were prepared using the same procedure as described in EXAMPLE 1 above for the labeled conjugates for Listeria. Affinity-purified goat antibodies to E. coli O157:H7 and Salmonella species were both obtained from Kirkegaard and Perry Laboratories Inc. of Gaithersburg, Md., and Fitzgerald, respectively. Each type of antibody was labeled with a 15-fold molar excess of Alexa-Fluor 647-succinimidyl ester and separated from the free dye by gel filtration as described above in EXAMPLE 1. After spectral characterization, the E. coli conjugate and Salmonella conjugates were diluted to 1.2 μg IgG/mL and 2.4 μg IgG/mL, respectively, in a buffer that included 50 mM Tris-HCl, pH 8.0, 54 mg BSA/mL, 18% sucrose, 200 mM NaCl, 0.5% Tween-20, and 5 mg of goat gamma globulin/mL. This solution is referred to herein as “Reagent B.”

Affinity-purified goat anti-E. coli O157:H7 and Salmonella antibodies were respectively biotinylated by reacting each antibody with a 10-15 fold molar excess of NHS-LC-LC-biotin as described in EXAMPLE 1. Six μL of a 0.5 μM solution of each biotinylated antibody was applied to separate spots of NeutrAvidin which had been previously applied and adsorbed to the surface of a polystyrene planar waveguide.

### EXAMPLE 10

The combined immunoassay was performed by harvesting E. coli O157:H7 or Salmonella cells grown on XLD agar from VWR International of West Chester, Pa., with 0.5 mL of Reagent B and slight agitation of the XLD agar surface for 30 seconds, collecting the Reagent B, and injecting 100 μL of the fluid into a cartridge containing the antibody-spotted planar waveguide. The cartridge was then placed in the BioCentrex portable analyzer and fluorescence emanating from each spot was monitored at 15-second intervals for a duration of ten minutes. The rates recorded by the analyzer are proportional to the concentration of E. coli O157:H7 and Salmonella harvested by Reagent B.

### EXAMPLE 11

### Combined Assay for E. coli O157:H7 and Salmonella typhimurium

E. coli O157:H7 (ATCC #700531) and Salmonella choleraesuis subspecies choleraesuis, subtype typhimurium (ATCC#14028) were diluted to about 40 cells/mL and about 20 cells/mL, respectively, in 1.0 mL of BPW. The diluted cells were incubated at ambient temperature for one hour. The entire cell incubation mixtures were then poured onto respective XLD agar plates and incubated for 4, 6 and 8 hours at 37° C. The plates were then harvested with Reagent B and tested on the portable analyzer. Binding rates from one to five minutes were assayed for both E. coli and Salmonella, grown on separate XLD agar plates and on combined plates. The data are shown in TABLE 7.

TABLE 7
Fluorescence Rates (1-5 Minutes)

| | Plated: | | | | | |
|---|---|---|---|---|---|---|
| | E. coli O157:H7 | | Salmonella | | Both | |
| Hours at 37 C. | O157:H7 | Sal | O157:H7 | Sal | O157:H7 | Sal |
| 4 | 11 | 11 | 15 | 17 | 1 | 14 |
| 6 | 254 | 18 | 10 | 96 | 250 | 82 |
| 8 | 1955 | 11 | 3 | 250 | 1459 | 223 |

The results that are set forth in TABLE 7 show that the technique of the present invention is useful for assaying both E. coli O157:H7 and Salmonella typhimurium individually and together. Furthermore, accurate results can be obtained after only about six hours of incubation.

EXAMPLES 1 through 11 show that teachings of the present invention may be used to effect rapid general and specific testing (i.e., testing without significant cross-over); that is, the ability to test for contamination by both total counts of microorganisms and the ability to test for the presence of specific microorganisms, respectively.

Moreover, large quantities of culture media need not be used or handled when teachings of the present invention are employed, as microorganisms of interest need not be proliferated to a visible state (e.g., colonies, plaques, cloudiness, etc.). Sample handling may also be reduced to a relatively small volume of liquid.

Sensitivity can be maximized when microorganisms are grown on semisolid growth media, as the microorganisms are a priori concentrated on the surfaces of such growth media.

Although the foregoing description contains many specifics, these should not be construed as limiting the scope of the present invention, but merely as providing illustrations of some of the presently preferred embodiments. Similarly, other embodiments of the invention may be devised which do not depart from the spirit or scope of the present invention. Features from different embodiments may be employed in combination. The scope of the invention is, therefore, indicated and limited only by the appended claims and their legal equivalents, rather than by the foregoing description. All additions, deletions and modifications to the invention as disclosed herein which fall within the meaning and scope of the claims are to be embraced thereby.

What is claimed:

1. A method for detecting microorganisms, comprising: obtaining a sample potentially containing at least one microorganism of interest;
applying at least a portion of the sample to a growth medium with at least some selectivity for the at least one microorganism of interest;
permitting the at least one microorganism to grow;
harvesting microorganism that may have been grown with the growth medium before or after microorganism growth is visibly evident; and
conducting an immunoassay for the at least one microorganism of interest on the potentially harvested microorganism.

2. The method of claim 1, wherein applying comprises applying at least a portion of the sample to an agar.

3. The method of claim 2, wherein harvesting includes applying a solution to the growth medium.

4. The method of claim 3, wherein applying the solution comprises applying a solution comprising at least one component of the immunoassay to the growth medium.

5. The method of claim 3, wherein harvesting further includes washing a surface of the agar with the solution.

6. The method of claim 3, wherein conducting the immunoassay comprises assaying the solution after harvesting microorganism from the growth medium.

7. The method of claim 1, wherein permitting is effected for a sufficient amount of time to increase a population of the at least one microorganism of interest to a detectable level.

8. The method of claim 1, wherein harvesting includes applying a solution to the growth medium.

9. The method of claim 8, wherein applying the solution to the growth medium comprises applying a solution including at least one component of the immunoassay to the growth medium.

10. The method of claim 8, wherein harvesting further includes washing the growth medium with the solution.

11. The method of claim 1, wherein conducting the immunoassay comprises conducting a fluorescence waveguide immunoassay.

12. The method of claim 11, wherein conducting the fluorescence waveguide immunoassay includes flowing a solution including harvested microorganism onto a waveguide surface.

13. The method of claim 12, wherein conducting the fluorescence waveguide immunoassay further includes determining a presence or absence of the at least one microorganism of interest in the solution.

14. The method of claim 1, further comprising:
repairing microorganisms present in the sample before permitting the at least one microorganism to grow.

15. The method of claim 1, wherein obtaining comprises obtaining a sample potentially containing a Listeria species.

16. The method of claim 15, further comprising:
repairing microorganisms present in the sample before permitting the at least one microorganism to grow.

17. The method of claim 16, wherein repairing comprises introducing the sample into a repair solution.

18. The method of claim 17, wherein introducing comprises introducing the sample into a repair solution comprising peptone water or Letheen broth.

19. The method of claim 16, wherein repairing occurs for about one hour to about four hours.

20. The method of claim 16, wherein repairing occurs for about two hours.

21. The method of claim 16, wherein applying the sample comprises applying at least some of the repair solution to the growth medium.

22. The method of claim 15, wherein applying comprises applying the sample to a growth medium comprising at least one of Oxford agar, modified Oxford agar, PALCAM agar, or Aloa agar.

23. The method of claim 15, wherein permitting comprises permitting any Listeria species of microorganism exposed to the growth medium to incubate for about twelve hours to about twenty four hours.

24. The method of claim 23, wherein permitting comprises permitting any Listeria species of microorganism exposed to the growth medium to incubate for about fourteen hours.

25. The method of claim 15, wherein permitting is effected at a temperature of about 37° C.

26. The method of claim 15, wherein conducting the immunoassay comprises applying harvested microorganism to a waveguide of a fluorescence waveguide immunoassay.

27. A system for facilitating growth and assay of microorganisms, comprising: a growth plate including:
a first receptacle configured to receive and contain a quantity of growth medium; and
a second receptacle adjacent to said first receptacle for receiving a harvest fluid from a surface of the growth medium.

28. The system of claim 27, further comprising:
a lid for covering the first and second receptacles of the growth plate.

29. The system of claim 28, wherein the lid includes an access hole for providing access to a portion of at least one of the first and second receptacles of the growth plate without requiring removal of the lid from the growth plate.

30. The system of claim 29, wherein the access hole is configured to reduce a potential for the introduction of contaminants into at least one of the first and second receptacles.

31. The system of claim 27, further comprising:
a lavage apparatus for suspending cells on a surface of growth medium within the first receptacle in a wash solution and causing at least some of the wash solution to enter the second receptacle.
